(19) United States
(12) Patent Application Publication
(10) Pub. No.: US 2009/0275670 A1
(43) Pub. Date: Nov. 5, 2009

(54) DOG PHEROMONE FORMULATION AND DELIVERY SYSTEM

(76) Inventor: GERALD L. MARSHALL, Athens, TX (US)

Correspondence Address: JACQUELINE S LARSON, 245 AVINGTON ROAD, ALAMEDA, CA, 94502, US

(21) Appl. No.: 12/430,075

(22) Filed: Apr. 25, 2009

Related U.S. Application Data
(60) Provisional application No. 61/125,977, filed on Apr. 30, 2008.

(51) Int. Cl. A61K 47/30; A61P 25/00

(52) U.S. Cl. 514/772.3

(57) ABSTRACT

The present invention is directed to a dog behavioral control system comprising a low melting point polymer or copolymer, an unsaturated long chain fatty alcohol, and a dog-appeasing pheromone to produce a solid slow release generator of the pheromone. The system is useful for making animal collars and the like.

### FIELD OF THE INVENTION

The present invention is directed to a dog behavioral control system comprising a low melting-point polymer or copolymer, an unsaturated long chain fatty alcohol, and a dog-appeasing pheromone to produce a solid slow release generator of the pheromone. The system of the invention releases the dog-appeasing pheromone efficiently and uniformly over an extended period of time of up to about four weeks or longer. The system is useful for the manufacture of animal collars and the like.

Some pet dogs exhibit stress-related behavior, which can take the form of, for example, being nervous of noises, timid with other dogs and/or with people, hyperactive in the house, constant barking, snappish toward owners or others, dislike of being touched, and the like. This behavior can become so bad that the dog will be given up by its owner and is often euthanized.

It is known that a mother dog produces a pheromone to calm and reassure her puppies. Dogs recognize this pheromone throughout life, as it appears to have a relaxing and calming effect on both the young and adults.

An artificial dog-appeasing pheromone (“DAPH”) has been developed, Pheromone “H”, which mimics the mother dog's calming pheromone. Products containing Pheromone “H” are available as a diffuser or from a spray bottle. However, such products are inconvenient in that, for example, they cannot easily accompany the dog everywhere and thus cannot consistently modify the animal's behavior. A DAPH delivery device that is always in the presence of the dog to provide a consistent and long-term treatment regimen is therefore needed. Additionally, Pheromone “H” is primarily a blend of natural fatty acids, which presents a unique challenge to the preparation of solid polymeric delivery devices, such as a pet collar, an ear tag or the like. Not only must the ingredients be processed at a lower temperature than is normally used in the art of polymeric extrusion, but the necessity for including high amounts of pheromone fatty acids in the polymeric article leads to undesirable results such as, for example, a residue of fatty acid oils on the surface, release rates of the pheromone that are unacceptably fast, articles that are too soft and can be pulled apart with tension, or polymeric material that cannot be extruded at either high or low temperatures.

### SUMMARY OF THE INVENTION

It has now been discovered by the applicant that by using an unsaturated long chain fatty alcohol together with a low melting-point polymer, one is able to obtain a polymeric system suitable for the controlled delivery of the dog-appeasing Pheromone “H” (“Pheromone H” or “DAPH”). More particularly, the present invention is directed to a solid dog behavioral control system comprising a dog control formulation comprising a low melting polymer or copolymer (that is, a polymer or copolymer having a melt temperature of below 250° F.), the dog-appeasing Pheromone “H”, and an unsaturated long chain fatty alcohol of from about 11 to about 18 carbon atoms. The system is useful for making articles or devices such as animal collars, tags, solid diffusers, and the like, which articles or devices are also encompassed by the present invention, for releasing the DAPH to a local environment in contact with the animal, over an extended or prolonged period of time. By “extended or prolonged period of time” is meant for a period of activity longer than the period of activity exhibited by the raw active ingredient alone. The extended period of release can be of up to four weeks or longer.

The present invention is further directed to a method for calming stress-related behavior in dogs, the method comprising placing a dog-appeasing device of the present invention into contact with the dog to be treated. The dog-appeasing device is capable of remaining with the animal at all times, such that the dog is in the continuous presence of the dog-appeasing pheromone for the useful life of the device; that is, for as long as an effective amount of the DAPH is released from the device, which can be of up to four weeks or longer.

The “fatty alcohol” or “solid fatty alcohol” useful in the present invention is any unsaturated fatty alcohol of from about 11 to about 18 carbon atoms and that is a solid at room temperature. Such fatty alcohols include, but are not limited to, cetyl alcohol (which includes: Cetanol, 1-Hexadecanol, Ethal, Ethol, Palmityl alcohol, Hexadecan-1-ol, Hexadecyl alcohol, Hexadecanol, Alcohol, C16, Atalco C, Cachalot C-50, Cetaffine, Cetal, Cetylol, CO-1670, Crodacol-cas, DYTOL F-11, Loroll 24, Loxanol K, and Product 308), stearyl alcohol (which includes: Octadecanol, Stenol, Octadecyl Alcohol, Decyl Octyl Alcohol, Stearol, N-octadecanol, Adol 68, Alfol 18, Atalco S, CO-1895, CO-1897, Crodacol-S, Dytol E-46, Lorol 28, Polaax, Sipol S, Siponol S, and Steraffine) and Oleyl Alcohol (which includes: Cis-9-Octadecen-1-01). In one aspect, the solid fatty alcohol is present in the formulation of the invention in an amount of from about 5 wt % to about 50 wt %. In another aspect, the solid fatty alcohol is present in an amount of from about 12 wt % to about 30 wt %. In a further aspect, the solid fatty alcohol is present in an amount of from about 12 wt % to about 25 wt %.

The “low melting polymer or copolymer” is selected from those polymers or copolymers that are a solid at room temperature and have a melt temperature of below 250° F. In one aspect, the low melting polymer or copolymer has a melt temperature of below 200° F. Examples of polymers and copolymers useful in the present invention include, but are not limited to, polyethylene, polyvinyl acetate, polyethylene, ethylene acid copolymers, ethylene acrylates, polyurethanes, styrene-butadiene, polyvinyl acetate, polyvinyl butyral, and mixtures and copolymers thereof. In one aspect, the low melting polymer or copolymer is present in the formulation of the invention in an amount of from about 40 wt % to about 85 wt %. In another aspect, it is present in an amount of from about 65 wt % to about 80 wt %. In a further aspect, the polymer or copolymer is present in an amount of from about 70 wt % to about 75 wt %.

Dog-appeasing Pheromone “H” is a proprietary composition (from Modern Veterinary Therapeutics, Florida) that comprises linoleic acid, capric acid, pentadecylic acid, azelaic acid, pimelic acid, lauric acid, myristic acid, cholesterol, C1-docosanol, squalene (shark origin) and BHT. The pheromone is volatile and will decompose at the high temperatures normally used to manufacture pet collars. Additionally, because Pheromone “H” is composed primarily of natural fatty acids, fatty acid oils interfere with the production, the physical characteristics and/or the aesthetics of normal collars. In one aspect, the DAPH is present in the formulation of the invention in an amount of from about 0.3 wt % to about 20 wt %. In another aspect of the invention, the DAPH is present in an amount of from about 3 wt % to about 15 wt %. In a further aspect, the DAPH is present in an amount of from about 5 wt % to about 10 wt %.

To prepare dog behavioral control systems according to the invention, the unsaturated long chain fatty alcohol is added to a low melting polymer or copolymer and mixed until uniformity is achieved. The pheromone is then added with mixing to make a blend. This blend formulation may then be processed into a shaped article or device, such as a pet collar or a tag or the like, on a conventional extruder or molding machine at low temperatures (that is, at a temperature that will melt the low melting polymer or copolymer, which is generally below about 250° F.) by methods known in the art.

Additional components may optionally be included in the dog behavioral control system of the invention. Such optional ingredients can include, but are not limited to, plasticizers, synergists, fragrances, coloring agents, preservatives, antioxidants, light stabilizers, and the like.

After being processed into the desired shape, the article or device, such as a collar, is placed into close proximity of a dog to be treated; that is, the device will be located, by attachment or other means, in sufficient proximity to or contact with the animal such that the DAPH will, together with the solid fatty alcohol, be released to the surface of the article as a result of the dog's body heat. The combination of the unsaturated long chain fatty alcohol and the low-melting polymer allows the pheromone to be released to the surface of the device continuously over a period of at least four weeks while it is simultaneously being transferred to the animal's hair and down to the skin.

The following examples illustrate the practice of the present invention but are not limiting. Parts are given as percentages and temperature are in degrees Fahrenheit unless otherwise noted.

### EXAMPLES

### Example 1

The formulation in Table 1 (below) is prepared, and is then formed into a dog collar, as follows:

TABLE 1

| Ingredients: | Percentages: |
|---|---|
| Pheromone “H” | 6.0 |
| Cetyl Alcohol | 20.0 |
| Polymer MU-76000 | 73.8 |
| Lime Green colorant | 0.2 |

### Sources:

Pheromone “H”—proprietary blend, Modern Veterinary Therapeutics.

Cetyl alcohol (1-hexadecanol)—Procter & Gamble.

Microthene® Polymer MU-76000 (ethylene-vinyl acetate copolymer, vinyl acetate content: 18%, ground powder, melt index: 32 g/10 min (EMI), avg. particle size: 35 mesh)—Equistar Chemicals, LP.

Colorant—Day Glow Color Corp.

### Mixing Procedure:

1. The polymer is weighed and placed into a mixing vessel.
2. The colorant is added to the polymer with mixing.
3. The cetyl alcohol is added to the polymer and mixed until uniform.
4. Optional Step: Fragrance is added to the Pheromone “H” active phase and mixed into the DAPH before adding to the polymer/fatty alcohol mixture. (This step is optional if fragrance is desired).
5. The Pheromone “H” (with optional fragrance) is added to the polymer/fatty alcohol while mixing, and mixing is continued for 1-2 hours.
6. The resulting blend is allowed to cool to room temperature, preferably overnight to allow for easy feeding of the blend while extruding.
   The blend is then extruded or molded, with temperature settings at 190° F. for zones 1, 2, 3 and 4, into the shape desired, which in this Example was a dog collar.

### Examples 2-6

Following the procedures of Example 1, dog collars were prepared from each of the formulations in Table 2, Table 3, Table 4, Table 5 and Table 6, below.

TABLE 2

| Ingredients: | Percentages: |
|---|---|
| Pheromone “H” | 6.0 |
| Cetyl Alcohol | 20.0 |
| Polymer MU-76000 | 70.8 |
| Lime Green colorant | 0.2 |
| Fragrance | 3.0 |

TABLE 3

| Ingredients: | Percentages: |
|---|---|
| Pheromone “H” | 6.0 |
| Cetyl Alcohol | 12.5 |
| Polymer MU-76000 | 78.3 |
| Lime Green colorant | 0.2 |
| Fragrance | 3.0 |

TABLE 4

| Ingredients: | Percentages: |
|---|---|
| Pheromone “H” | 9.0 |
| Cetyl Alcohol | 20.0 |
| Polymer MU-76000 | 70.8 |
| Lime Green colorant | 0.2 |

TABLE 5

| Ingredients: | Percentages: |
|---|---|
| Pheromone “H” | 9.0 |
| Cetyl Alcohol | 20.0 |
| Polymer MU-76000 | 67.8 |
| Lime Green colorant | 0.2 |
| Fragrance | 3.0 |

TABLE 6

| Ingredients: | Percentages: |
|---|---|
| Pheromone “H” | 9.0 |
| Cetyl Alcohol | 12.5 |
| Polymer MU-76000 | 75.3 |
| Lime Green colorant | 0.2 |
| Fragrance | 3.0 |

### Example 7

The collars of Example 1 were subjected to efficacy evaluation. The collars were placed onto dogs that had stress-related behavioral problems, such as being nervous of noises, timid with other dogs and people, hyperactive in the house, barked at everything, shied away from other dogs, too playful, chewed a lot, destructive while in the house, very snappy with owners, did not like to be fussed with or touched, and/or constantly barked at owners for attention. The dogs remained in their home environments for the period of the test.

After the DAPH collars were applied to the dogs, 80% of the treated dogs experienced improvements: Some dogs were more relaxed, trembling stopped, confidence was restored, hyperactivity stopped, great improvement when dealing with others, dogs appeared happier, and excessive barking stopped.

At the end of four weeks the collars were removed from the dogs, after which a majority of the dogs reverted back to being nervous (trembling) and timid, were not so confident, were hyperactive and started back to barking a lot.

During the 3-month testing period, visible evidence was observed as to the functionality of the DAPH collars. The majority of the testing subjects exhibited positive results after using the collar and most regressed to their previous states once its use was suspended. Most subjects' owners expressed a desire to purchase the collars.

What is claimed is:

1. A polymeric formulation comprising a low melting polymer or copolymer, an unsaturated long-chain fatty alcohol of from about 11 to about 18 carbon atoms, and a dog-appeasing pheromone.

2. A polymeric formulation according to claim 1 wherein the dog-appeasing pheromone is Pheromone “H”.

3. A polymeric formulation according to claim 1 wherein the low melting polymer or copolymer is ethylene-vinyl acetate copolymer.

4. A polymeric formulation according to claim 1 wherein the long-chain fatty alcohol is cetyl alcohol, stearyl alcohol or oleyl alcohol.

5. A polymeric formulation according to claim 1 which comprises from about 40 wt % to about 85 wt % of a low melting polymer or copolymer, from about 5 wt % to about 50 wt % of an unsaturated long-chain fatty alcohol and from about 0.3 wt % to about 20 wt % of a dog-appeasing pheromone.

6. A polymeric formulation according to claim 5 wherein the dog-appeasing pheromone is Pheromone “H”.

7. A polymeric formulation according to claim 5 wherein the low melting polymer or copolymer is ethylene-vinyl acetate copolymer.

8. A polymeric formulation according to claim 5 wherein the long-chain fatty alcohol is cetyl alcohol, stearyl alcohol or oleyl alcohol.

9. A polymeric formulation according to claim 1 which comprises from about 65 wt % to about 80 wt % of a low melting polymer or copolymer, from about 12 wt % to about 30 wt % of an unsaturated long-chain fatty alcohol and from about 5 wt % to about 10 wt % of a dog-appeasing pheromone.

10. A polymeric formulation according to claim 9 wherein the dog-appeasing pheromone is Pheromone “H”.

11. A polymeric formulation according to claim 9 wherein the low melting polymer or copolymer is ethylene-vinyl acetate copolymer.

12. A polymeric formulation according to claim 9 wherein the long-chain fatty alcohol is cetyl alcohol, stearyl alcohol or oleyl alcohol.

13. A polymeric formulation according to claim 9 wherein the low melting polymer or copolymer is ethylene-vinyl acetate copolymer, the long-chain fatty alcohol is cetyl alcohol, and the dog-appeasing pheromone is Pheromone “H”.

14. A dog behavioral control system comprising a polymeric formulation that comprises a low melting polymer or copolymer, an unsaturated long-chain fatty alcohol of from about 11 to about 18 carbon atoms, and a dog-appeasing pheromone.

15. A dog behavioral control system according to claim 14 wherein the low melting polymer or copolymer is ethylene-vinyl acetate copolymer, the long-chain fatty alcohol is cetyl alcohol, stearyl alcohol or oleyl alcohol, and the dog-appeasing pheromone is Pheromone “H”.

16. A dog behavioral control system according to claim 15 wherein the polymeric formulation comprises from about 40 wt % to about 85 wt % of ethylene-vinyl acetate copolymer, from about 12 wt % to about 30 wt % of cetyl alcohol and from about 5 wt % to about 10 wt % of the dog-appeasing pheromone Pheromone “H”.

17. A dog behavioral control system according to claim 14 which is a device in the shape of an animal collar.

18. A dog behavioral control system according to claim 16 which is a device in the shape of an animal collar.

19. A method for calming stress-related behavior in dogs over an extended period of time, the method comprising placing a dog behavioral control system into close proximity with a dog to be treated, the dog behavioral control system comprising a polymeric formulation that comprises a low melting polymer or copolymer, an unsaturated long-chain fatty alcohol of from about 11 to about 18 carbon atoms, and a dog-appeasing pheromone.

20. A method according to claim 19 wherein the dog behavioral control system is a device in the shape of an animal collar.
